(19) United States
(12) Patent Application Publication
(10) Pub. No.: US 2026/0337573 A1
(43) Pub. Date: Oct. 29, 2026

(54) METHODS AND PRODUCTS FOR MEAT CELL CULTURE

(71) Applicant: QUEST MEAT LTD, Birmingham West Midlands (GB)

(72) Inventors: Ivan Wall, Birmingham West Midlands (GB); John Sinden, Birmingham West Midlands (GB); Petra Hanga, Birmingham West Midlands (GB); Miguel Ferreira, Birmingham West Midlands (GB); Debora Oliveira, Birmingham West Midlands (GB)

(21) Appl. No.: 19/113,380

(22) PCT Filed: Nov. 8, 2023
(86) PCT No.: PCT/GB2023/052921

§ 371 Date: Mar. 19, 2025

(30) Foreign Application Priority Data
Nov. 8, 2022 (GB) 2216606.0

(51) Int. Cl. C12N 5/077; A23L 13/00; C12N 1/185

(52) U.S. Cl. CPC C12N 5/0658; A23L 13/00; C12N 1/185; C12N 2500/74; C12N 2511/00; C12N 2513/00; C12N 2531/00

(57) ABSTRACT

Provided herein are methods for culturing animal cells in a culture medium comprising a culture ingredient that is composed of or produced by yeast. In particular, the culture ingredient comprises, consists essentially of or, or consists of, inactivated yeast. Also provided herein is use of inactivated yeast as a matrix, support, carrier or microcarrier for growing meat cells in in vitro culture.

### FIELD OF THE INVENTION

This invention relates to a method for growing animal cells to enhance the production of cultivated meat. The invention also relates to products for use in these methods, and products that result from these methods.

### Background to the Invention

Humans currently consume half a trillion tonnes of meat per year. Highly intensive, industrial farming used to meet this demand is a major contributor to climate change, causing significant environmental damage, human disease, and animal cruelty. Current levels of meat consumption are not sustainable, yet they are set to double by 2050. A whole new industry sector is emerging, known as alternative proteins. The alternative proteins sector is focused on creating proteins as foods using alternative approaches to industrial farming to meet global food security demands. Alternative proteins include plant-based meats, fermentation products such as milk substitutes and cultivated meat. Cultivated meat companies are now undertaking R&D to create meat from animal-derived stem cells.

There are numerous benefits to cultivated meat in comparison to farmed meat. Cultivated meat will theoretically use 95% less land and 78% less water, whilst producing 92% fewer carbon emissions. However, the single major challenge facing cultivated meat companies is how to manufacture at large scale cost-effectively. For the cultivated meat industry to be commercially viable, the price per kg of meat product needs to reach a similar level to that of existing organic meat. To achieve cost equivalence with current sustainably produced meat, innovative tools are needed to create radical process improvements that will increase meat product yield whilst addressing process economics. The present invention proposes a method for growing animal cells using an ingredient that will support meat cell culture.

The cell types used to create or develop the creation of cultivated meat (meat cells), including myosatellite cells, myoblasts, mesenchymal stem cells, and adipose tissue-derived mesenchymal stem cells are adherent cells that cannot be grown in suspension culture without the need for microcarriers or the generation of large spheroids. Microcarriers are small spheres that can be placed in suspension culture such as large fermentation tanks or bioreactors that provide a physical attachment surface for growth of meat cells in suspension. Microcarriers optimised for cell growth have been made from plastic materials but these are not suitable for meat production as the plastic cannot form part of the final edible product, and downstream removal of the plastic before creating meat from the cells is inefficient. There have been developments leading to edible microcarrier production from hydrogel materials with RGD peptide sequences that promote cell attachment (U.S. Pat. No. 9,752,122B2 Edible and animal-product-free microcarriers for engineered meat). These are expensive and difficult to produce in large quantities to make meat.

### SUMMARY OF THE INVENTION

The invention provides methods, uses and products relating to cultured meat products.

In one aspect the present invention proposes a method for growing adherent meat cells using an edible ingredient means that supports cell culture on flat surfaces and in 3D environments including bioreactors and other cell culture devices.

In one aspect the present invention proposes a method for growing adherent meat cells using an edible ingredient means that supports cell culture on flat surfaces.

In one aspect the present invention proposes a method for growing adherent meat cells using an edible ingredient means that supports cell culture in 3D environments.

The ingredient means comprises or is a derivative of or is a secreted product of microorganisms including fungi and bacteria to produce an edible material that can enable cell attachment, and/or growth and/or differentiation via physical and/or nutritional support for cultivated meat applications. Typically, the microorganisms are single-celled microrganisms.

The ingredient means is preferably provided by fungi in the form of yeast or filamentous fungi species that is used to support the growth of animal cells in dishes, flasks and bioreactors.

The ingredient means may comprise, consist essentially of, or consist of, inactivated yeast. Inactivated yeast is known in the art and is commercially-available, and is sometimes referred to as nutritional yeast. The inactivated yeast typically comprises, consists essentially of or consists of Saccharomyces cerevisiae, also known as baker's yeast or brewer's yeast. The inactivated yeast may typically be in the form of flakes or a powder. Other yeasts that may be used include, for example, Pichia pastoris and Schizosaccharomyces pombe.

The ingredient means can be grown in a nutritional medium, and retrieved through flocculation and filtration and then dried at high temperatures or freeze dried to inactivate it. In some embodiments the ingredient means (sometimes referred to herein simply as “the ingredient”) is produced by growing a microorganism in a culture medium, typically a nutritional medium. The ingredient can be retrieved or harvested by flocculation and filtration and then dried at high temperatures or freeze dried. The drying step typically inactivates some, most, substantially all or all of the microorganism cells. For example, the drying step may inactivate 20% or more, 50% or more, 80% or more, or 95% or more-for example 99% or more-of the cells subjected to the drying (inactivation) step.

As used herein, a nutritional medium is any medium in which the microorganism, for example yeast such as Saccharomyces cerevisiae, can grow and optionally proliferate. Such media typically comprise at least one carbohydrate source, typically at least one sugar, for example glucose, dextrose or sucrose. The nutritional medium may comprise one or more amino acids. Yeast growth media are known in the art, and can be used as the nutritional medium. The nutritional medium may include, or alternatively explicitly exclude, peptone. Peptone is well-known in the art, and is a mixture of peptides and free amino acids typically obtained from pancreatic hydrolysis of animal tissue. In some embodiments, the nutritional medium is a synthetic medium, for example a synthetic minimal medium or a synthetic complete medium, as is known in the art.

Examples of well-known media in which yeast can be grown, include YPD, medium, YPG medium and YPAD medium.

The nutritional medium may be a liquid (e.g. broth) or solid (e.g. agar) form. Typically it is a liquid.

In some embodiments, the ingredient is produced in a nutritional medium, is retrieved at the end of culture through flocculation and filtration, and is then dried at high temperatures or freeze dried to inactivate cells and produce either flakes or powder.

The ingredient means can be obtained via additional methods, including extraction from waste products from breweries, cheese factories, whisky distillers and cider factories.

Further embodiments include the extraction of substances from fungi including chitin and its use as a purified material as an additive and microcarriers. In some embodiments, the substances from fungi are extracted and used as a purified material as an additive and cell attachment substrate.

The invention relates generally to the culturing of meat cells.

The meat cells that are cultured are typically muscle cells and/or fat cells, or undifferentiated cells capable of differentiating into muscle cells and/or fat cells.

In some embodiments, the meat cells that are cultured are muscle cells. In some embodiments, the meat cells that are cultured are fat cells. In some embodiments, the meat cells that are cultured are a co-culture of muscle cells and fat cells. In some embodiments, the meat cells that are cultured are capable of differentiating into muscle cells. In some embodiments, the meat cells that are cultured are capable of differentiating into fat cells. In some embodiments, the meat cells that are cultured comprise cells capable of differentiating into fat cells and cells capable of differentiating into fat cells. In some embodiments, the meat cells that are cultured are muscle-derived cells and/or fat-derived cells.

In some embodiments the cells are myosatellite cells, myoblasts, mesenchymal stem cells or adipose tissue-derived mesenchymal stem cells, or any combination thereof.

In some embodiments, the undifferentiated cells capable of differentiating into muscle cells and/or fat cells are stem cells or precursor cells. In some embodiments, the undifferentiated cells capable of differentiating into muscle cells and/or fat cells are stem cells, optionally pluripotent stem cells, for example induced pluripotent stem cells. In some embodiments, the stem cells are multipotent. In some embodiments, the stem cells are unipotent. In some embodiments, the stem cell is ectodermal. In some embodiments, the stem cell is mesodermal. In some embodiments, the stem cell is endodermal. In some embodiments, the cells are myosatellite cells, myoblasts, mesenchymal stem cells or adipose tissue-derived mesenchymal stem cells, or induced pluripotent stem cells, or any combination thereof.

In a further aspect, the invention provides the use of inactivated yeast as a matrix, support, carrier or microcarrier for growing meat cells in in vitro culture. The inactivated yeast typically comprises, consists essentially of or consists of Saccharomyces cerevisiae. The meat cells typically comprise or consist of muscle cells and/or fat cells. In some embodiments, meat cells typically comprise or consist of muscle-forming cells. In some embodiments, meat cells typically comprise or consist of fat-forming cells. In some embodiments, meat cells typically comprise or consist of muscle-forming cells or fat-forming cells.

In a further aspect, the invention provides a cultured meat product obtainable or obtained by the method of the first aspect or any other aspect described above and herein.

### BRIEF DESCRIPTION OF THE DRAWINGS

The invention will now be described solely by way of example and with reference to the accompanying drawings in which:

FIG. 1 shows growth of myogenic stem cells in low attachment plates without ingredient JP, or with ingredient JP.

FIG. 2 shows JP significant expansion of myoblast-derived muscle microtissues in 100 ml stirred culture confirmed by metabolic activity. (A) Bovine myosatellite cells cultured in a stirred culture vessel at 100 mL scale on ingredient-derived microcarriers. (B) Fluorescent images of live cells stained with calcein-AM showing as green taken at days 2 and 16 in culture. (C) Metabolic activity measured at different times in culture. Data shown as mean ±SD (N=3). Surprisingly, between day 2 and day 7 of culture in the presence of ingredient JP, metabolic activity of animal cells increased by about 2-fold. Furthermore, over a period of fourteen days in the presence of ingredient JP, metabolic activity of animal cells increased by about 10-fold. FIG. 3 shows expansion of muscle-derived stem cells as disperse uniform cells and microtissues. Over a period of eight days in the presence of ingredient JP, metabolic activity of animal cells increased by about 3-fold.

FIG. 4 shows adipose-derived stem cells growing as disperse single cell suspensions with uniform exposure to nutrients and oxygen. Culturing fat cells with the ingredient allows for their growth and/or propagation and/or proliferation and/or differentiation.

FIG. 5 shows myoblast cells cultured in a serum-based medium on multiple commercial brands of ingredient-derived microcarriers. Live cells are stained with calcein-AM and appear white. Images taken at days 2 and 13 in culture show cell growth and/or propagation and/or proliferation and/or differentiation on all commercial brands tested. Cultures using brands B and D showed increased amounts of fluorescent cells than cultures using brands A and C, indicating more live muscle cells.

FIG. 6 shows growth and/or propagation and/or proliferation and/or differentiation over time of myoblasts from different species on ingredient-derived microcarriers. Species shown here are bovine, mouse, and water buffalo, but these are not exhaustive. Live cells appear white and are stained with calcein-AM.

FIG. 7 shows growth and/or propagation and/or proliferation and/or differentiation of myoblast cells on ingredient-derived microcarriers when cultured in serum-based media (GM) or in serum-free media formulation (SF2). (A) Metabolic data measured at day 14 in culture. Data shown as mean ±SD (N=3). Surprisingly, myoblasts cultured in serum-free media showed about 3.5-fold increase in metabolic activity compared to myoblasts cultured in serum-based media. (B) Fluorescent images taken at day 14 in culture. Live cells appear white and are stained with calcein-AM.

FIG. 8 shows growth and/or propagation and/or proliferation and/or differentiation of animal cells cultured on ingredient-derived microcarriers in a serum-free media formulation up to 15 days. (A) shows a fluorescent image of live bovine myosatellite cells stained with calcein-AM (white). (B) shows metabolic activity of the cells described in part (A) measured at days 4 and 15, respectively. Data shown as mean ±SD (N=3). Metabolic activity of bovine myosatellite cells increased about 1.5-fold from day 4 to day 15. (C) shows a fluorescent image of live adipose-derived stem cells stained with calcein-AM (white). (D) shows metabolic activity of the cells described in part (C) measured at days 4 and 14, respectively. Data shown as mean ±SD (N=3). Metabolic activity of adipose-derived stem cells increased about 3-fold from day 4 to day 14.

FIG. 9 shows long term culture and growth and/or propagation and/or proliferation and/or differentiation of myosatellite cells on ingredient-derived microcarriers in stirred vessels of different scales in serum-based media up to 55 days. Fluorescent images taken of live cells (white) stained with calcein-AM at different time points in culture and metabolic activity measured at different time points. Data shown as mean ±SD (N=3). At each time point sampled, metabolic activity of the cells continued to increase. This indicates successful culture of the cells. From day 15 to day 47 in culture, the metabolic activity of myosatellite cells cultured on ingredient-derived microcarriers in serum-based media in stirred vessels increased by about 6-fold.

FIG. 10 shows a comparison of myosatellite cell culture on ingredient-derived microcarriers in serum-based media when using a commercial ingredient or an ingredient derived from brewery waste. Both commercial and brewery waste ingredients supported myosatellite cell growth and/or propagation and/or proliferation and/or differentiation. (A) Metabolic activity measured at day 7 in culture. Data shown as mean ±SD (N=3). (B) Fluorescent images taken at day 7 of live cells (white) stained with calcein-AM.

FIG. 11 shows a comparison of myosatellite cell culture on ingredient-derived microcarriers in serum-based media when using a commercial ingredient, ingredient derived from brewery waste and ingredient from brewery waste mixed with alginate. Fluorescent images taken at days 6 and 12 of live cells (white) stained with calcein-AM.

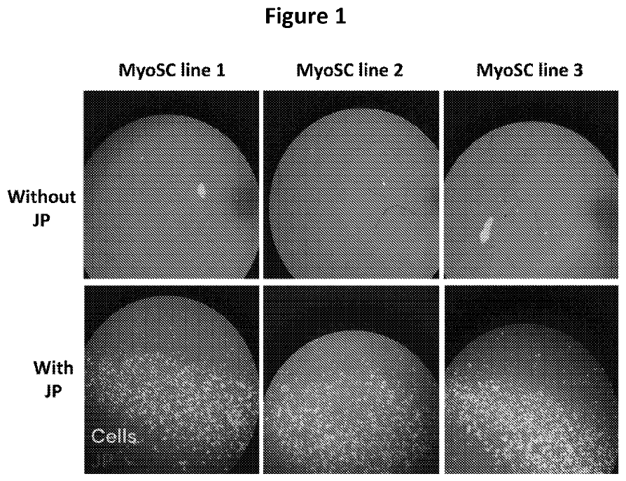

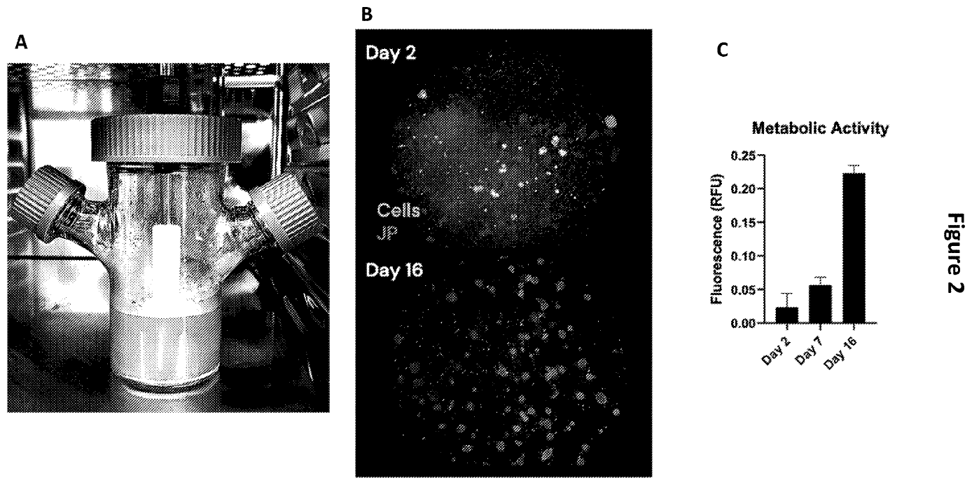

### DETAILED DESCRIPTION

The invention relates generally to methods for culturing animal cells using a culture ingredient that is composed of or produced by yeast cells. Such methods are particularly useful for producing edible material to be consumed by humans or animals as food.

Cell culture is used to create different products including medicines, food & drink and various industrial materials. The invention described herein generally provides a platform for growing animal-derived cells in culture using raw cell culture materials that can be processed into solid, semi-solid and liquid formats that which may be circular economy-derived. The platform enables cell growth, expansion and maturation to at least the same standard as current gold standard approaches.

A particular advantage of the technology described herein, is that yeast is an abundant waste product of fermentation in existing processes for making edible products, for example alcohol brewery or distillation. Furthermore, such yeast is already food-grade. Therefore, the re-use of yeast that has already been used in one food-grade process, is highly efficient.

Unless otherwise defined herein, technical and scientific terms used herein have the same meaning as commonly understood by one of ordinary skill in the art to which this application belongs and as commonly used in the art to which this application belongs.

### Cell Culture

Cell culture methods as described herein typically refer to in vitro or ex vivo methods of culturing non-human animal cells. In some embodiments, cell culture media comprises serum. In other embodiments, which are often preferred, the cell culture media does not comprise serum.

As will be understood by those skilled in the art, methods of cell culture typically refer to one or more of growth, propagation, proliferation, and differentiation of a cell under controlled conditions. In some embodiments, the method of culturing animal cells comprises growing the animal cells. In some embodiments, the method of culturing animal cells comprises propagating the animal cells. In some embodiments, the method of culturing animal cells comprises proliferating the animal cells. In some embodiments, the method of culturing animal cells comprises differentiating the animal cells.

It will be understood that different culture conditions can be used to promote proliferation over differentiation. For example, to increase the number of cells in a culture, conditions for proliferation may be favoured. Once an increased number of cells is obtained, different culture conditions may then be used to promote differentiation of the cells into one or more desired cell lineages.

### Animal Cells

As will be understood from the context of the invention, “animal cell” typically refers to a non-human animal cell. Animal cells for use in a cell culture method as described herein are typically obtained or are obtainable from a non-human animal.

In some embodiments, the non-human animal is a vertebrate or invertebrate.

In some embodiments, the vertebrate is a mammal, fish, amphibian, reptile, or bird.

In some embodiments, the mammal is a cow, pig, sheep, goat, deer, or rabbit. In some embodiments, the mammal is a cow, pig, sheep, goat, deer, rabbit, bison, buffalo, water buffalo, yak, llama, bat, donkey, horse, kangaroo, monkey, boar, whale, dolphin, or seal.

In some embodiments, the deer is an elk, fallow deer, moose, red deer, white-tailed deer, or reindeer.

In some embodiments, the bird is a chicken, duck, or turkey. In some embodiments, the bird is a chicken, duck, turkey, goose, quail, pigeon, guineafowl, ostrich, emu, or peacock.

In some embodiments, the fish is a barramundi, mackerel, herring, anchovy, sardine, carp, catfish, cod, seabream, Dentex, haddock, tuna, salmon, flounder, halibut, milkfish, rabbitfish, seabass, tilapia, trout, turbot, or Wuchang bream.

In some embodiments, the invertebrate is an insect, crustacean, or mollusc.

In some embodiments, one or more animal cells are co-cultured. In other embodiments, animal cells are cultured separately.

### Meat Cells

Meat typically refers to edible animal flesh. Typically, meat is comprises of one or more animal cell types. Meat includes, but is not limited to, 1, 2, 3 or all four of muscle cells, fat cells, connective tissue cells and blood cells.

In some embodiments, meat cells comprise or consist of muscle cells. In some embodiments, the muscle cells comprise progenitor muscle cells and/or differentiated muscle cells. In some embodiments, the muscle cells comprise one or more of myosatellite cells, myogenic stem cells, myoblasts, and myocytes. In some embodiments, the muscle cells comprise or consist of myosatellite cells. In some embodiments, the muscle cells comprise or consist of myogenic stem cells. In some embodiments, the muscle cells comprise or consist of myoblasts. In some embodiments, the muscle cells comprise or consist of myocytes. In some embodiments, muscle cells are proliferated using a cell culture method described herein. In some embodiments, myosatellite cells are proliferated using a cell culture method described herein. In some embodiments, myoblasts are proliferated using a cell culture method described herein. In some embodiments, muscle cells are differentiated using a cell culture method described herein. In some embodiments, myoblasts are differentiated into myocytes.

In some embodiments, meat cells comprise or consist of fat cells. In some embodiments, the fat cells comprise progenitor and/or differentiated cells. In some embodiments, the fat cells comprise one or more of adipose-tissue derived stem cells, adipose cells, and adipose-tissue derived mesenchymal stem cells. In some embodiments, the fat cells comprise adipose-tissue derived stem cells. In some embodiments, the fat cells comprise adipose cells. In some embodiments, fat cells are proliferated using a cell culture method described herein. In some embodiments, adipose-tissue derived stem cells are proliferated using a cell culture method described herein. In some embodiments, adipose-tissue derived mesenchymal stem cells are proliferated using a cell culture method described herein. In some embodiments, fat cells are differentiated using a cell culture method described herein. In some embodiments, adipose-tissue derived stem cells are differentiated into adipose cells. In some embodiments, the fat cells comprise or consist of brown fat cells. In some embodiments, the fat cells comprise or consist of white fat cells. In some embodiments, the fat cells comprise brown fat cells and white fat cells.

In some embodiments, meat cells comprise or consist of connective tissue cells. In some embodiments, the connective tissue cells comprise progenitor and/or differentiated cells. In some embodiments, the connective tissue cells comprise mesenchymal stem cells. In some embodiments, the connective tissue cells comprise fibroblasts. In some embodiments, connective tissue cells are proliferated using a cell culture method described herein. In some embodiments, connective tissue cells are differentiated using a cell culture method described herein.

In some embodiments, meat cells comprise or consist of blood cells. In some embodiments, the blood cells comprise progenitor and/or differentiated cells. In some embodiments, blood cells are proliferated using a cell culture method described herein. In some embodiments, blood cells are differentiated using a cell culture method described herein.

In some embodiments, one or more meat cells are co-cultured. In other embodiments, meat cells are cultured separately. In some embodiments the co-culture comprises, for example, meat cells and fat cells. In some embodiments the co-culture comprises, for example, meat cells and blood cells. In some embodiments the co-culture comprises, for example, meat cells and fat cells and blood cells.

Typically, animal cells may first be cultured to favour proliferation, to produce enough animal cells to form a meat product at a scale suitable for commercial production of edible meat products.

In some embodiments, once an appropriately high number of animal cells is obtained, different culture conditions may then be used to promote differentiation of the cells into one or more desired cell lineages. Typically, the one or more desired cell lineages comprise those typically identifiable in meat, i.e., meat cells. It will be understood that the one or more desired cell lineages may differ depending on the consumer meat product to be produced. In some embodiments, the one or more desired cell lineages comprise muscle cells and/or fat cells and/or connective tissue cells and/or blood cells.

### Ingredient

The invention relates to the use of a beneficial ingredient in meat cell culture. This ingredient may in some embodiments be viewed as a culture supplement.

In particular, the invention relates to methods of cell culture using an ingredient that is composed of or produced by a microorganism, typically a unicellular microorganism, more typically yeast.

The ingredient may alternatively or additionally comprise one or more molecules secreted or produced by yeast cells. As will be understood according to the present disclosure, the ingredient is typically a culture component or supplement.

In some embodiments, the yeast comprises budding yeast. In some embodiments, the budding yeast comprises one or more Saccharomyces yeast species. In some embodiments, the Saccharomyces yeast species comprises Saccharomyces cerevisiae. Many strains of Saccharomyces cerevisiae are known in the art, which can generally be used in the present invention in any combination. In some embodiments, the Saccharomyces yeast species comprises Saccharomyces pastorianus. In some embodiments, the Saccharomyces yeast species comprises Saccharomyces carlsbergensis.

In some embodiments, the yeast comprises fission yeast. In some embodiments, the fission yeast comprises Schizosaccharomyces yeast species. In some embodiments, the Schizosaccharomyces yeast species comprises Schizosaccharomyces pombe.

In the food and drink industries, yeast is often considered a byproduct of fermentation, such as in the production of beers, wines and spirits. For example, yeast is used in alcoholic fermentation, which is part of the process in producing alcoholic products including brewed or distilled alcoholic products. Accordingly, such yeasts are referred to in the art as brewer's yeast or distiller's yeast, respectively.

In the fermentation, brewing and distilling industries, after its use in fermentation, most of the yeast is typically removed by filtration and is considered a waste product. Surprisingly, the inventors demonstrate the utility of such yeast in methods for culturing animal cells. Use of such yeast is particularly beneficial in methods of cell culture described herein because the yeast is food-grade. Furthermore, such yeast is inexpensive.

In some embodiments therefore, raw substrate materials for the ingredient may be sourced from brewing and food production industries. In some embodiments, the raw substrate materials may be circular economy-derived.

In some embodiments, the raw substrate materials (typically yeast ingredient such as an inactivated yeast ingredient) may be processed into solid, semi-solid and soluble formats of ingredient that support cells in culture. In some embodiments, the raw substrate materials (typically yeast ingredient such as an inactivated yeast ingredient) may be processed into solid, formats of ingredient that support meat cells in culture. In some embodiments, the raw substrate materials (typically yeast ingredient such as an inactivated yeast ingredient) may be processed into semi-solid formats of ingredient that support meat cells in culture. In some embodiments, the raw substrate materials (typically yeast ingredient such as an inactivated yeast ingredient) may processed into soluble formats of ingredient that support meat cells in culture.

One or more species of yeast can be utilised in the production of a fermented product. It will therefore be understood that the ingredient for use in meat cell culture as described herein, may comprise one or more species of yeast. Yeast obtained from such sources may be treated before use in the methods described herein. In some embodiments, the yeast is washed. In some embodiments, the yeast is washed with water. In some embodiments, the yeast is washed with a food grade solvent. In some embodiments, the food grade solvent comprises ethanol. In some embodiments, the food grade solvent comprises isopropanol. In some embodiments, the food grade solvent comprises methanol. In some embodiments, the washing is carried out under static conditions. In some embodiments, the washing is carried out under agitated conditions.

In some embodiments, the yeast comprises baker's yeast. Such yeast can be purchased commercially and used in methods according to the present invention.

In some embodiments, a proportion of the yeast cells for use in the methods described herein are inactive. Methods of inactivating yeast are described in the art, including but not limited to inactivation by heat treatment (hot or cold) or by addition of organic acids.

In some embodiments, the yeast is inactivated by heat treatment. In some embodiments, the yeast is inactivated by treatment at a temperature of about −80 °C to about 0 °C or about 40 °C to about 150 °C. In some embodiments, the yeast is inactivated by heating to about 65 °C to about 150 °C, for example about 70 °C to about 100 °C, or about 70 °C to about 80 °C for example around 75 °C. The heat is typically applied long enough to rupture the cell membranes of the desired proportion of yeast cells, which may be heating to the stated temperature for at least one second, at least ten seconds, at least 30 seconds or at least one minute. An exemplary heat inactivation is around 10 seconds at 75 °C

In some embodiments, the yeast is inactivated by cooling the yeast to about −80 °C to about −20 °C. The freezing temperature is typically applied long enough to rupture the cell membranes of the desired proportion of yeast cells, which may be cooling to the stated temperature for at least one second, at least ten seconds, at least 30 seconds or at least one minute.

In some embodiments the yeast is inactivated by freeze-drying, also known as lyophilisation, which is a well-known technique in the art.

In some embodiments, the yeast is inactivated by addition of organic acids.

The ingredient can be supplied to the meat cell culture in different physical formats. In some embodiments, the inactivated yeast is provided to the cell culture as flakes. In some embodiments, the inactivated yeast is provided to the cell culture as particles. In some embodiments, the inactivated yeast is provided to the cell culture as a powder.

In some embodiments, the inactive yeast comprises dead yeast cells. In some embodiments, the inactive yeast comprises lysed yeast cells. In some embodiments, a majority of the yeast cells are inactivated. In some embodiments, about 80% or more of the yeast cells are inactivated. In some embodiments, about 85% or more of the yeast cells are inactivated. In some embodiments, about 90% or more of the yeast cells are inactivated. In some embodiments, about 91% or more of the yeast cells are inactivated. In some embodiments, about 92% or more of the yeast cells are inactivated. In some embodiments, about 93% or more of the yeast cells are inactivated. In some embodiments, about 94% or more of the yeast cells are inactivated. In some embodiments, about 95% or more of the yeast cells are inactivated. In some embodiments, about 96% or more of the yeast cells are inactivated. In some embodiments, about 97% or more of the yeast cells are inactivated. In some embodiments, about 98% or more of the yeast cells are inactivated. In some embodiments, about 99% or more of the yeast cells are inactivated. In some embodiments, about 90% or more of the yeast cells are inactivated.

To be cultured successfully, animal cells typically require nutritional support. In some embodiments, the inactive yeast provides nutritional support for animal cells or meat cells. In some embodiments, the inactive yeast comprises fibre. In some embodiments, the inactive yeast comprises amino acids, typically essential amino acids. In some embodiments, the inactive yeast comprises vitamin B.

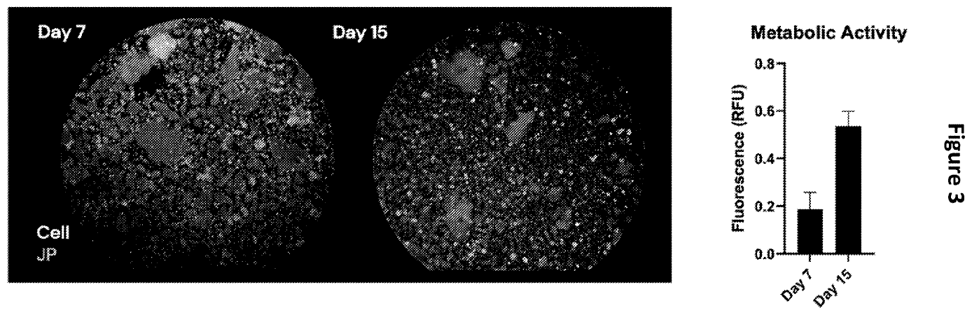

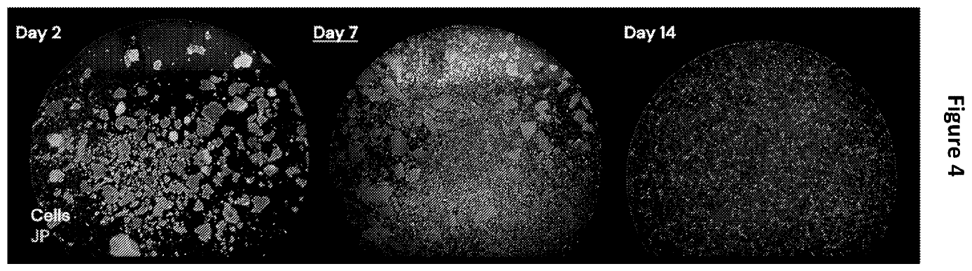

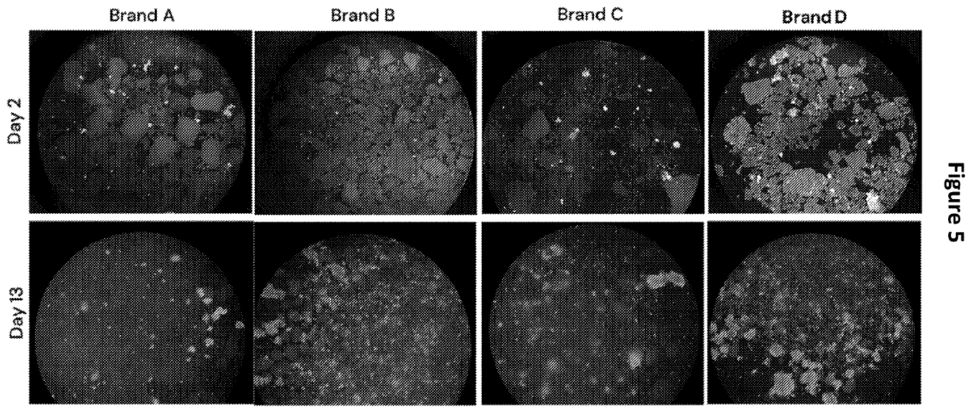

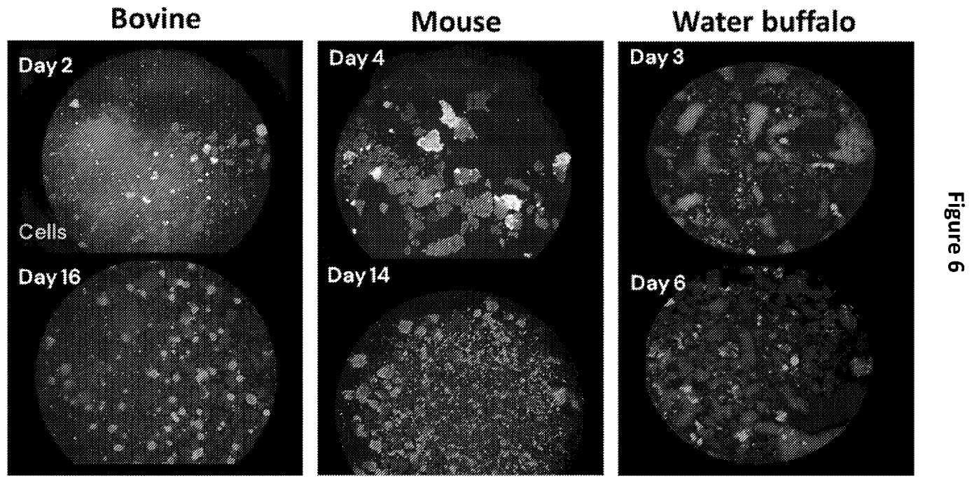

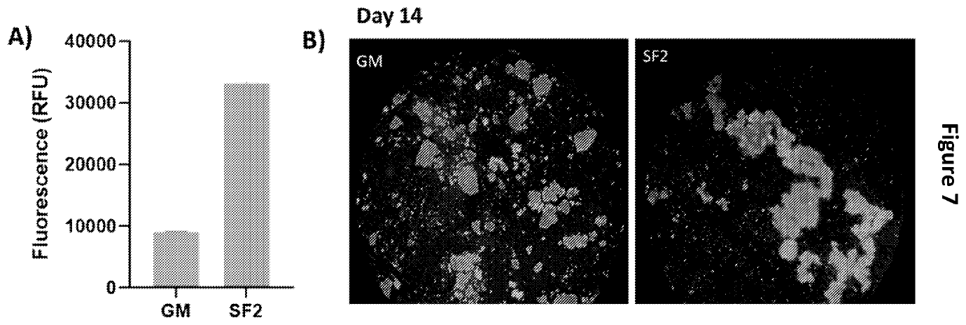

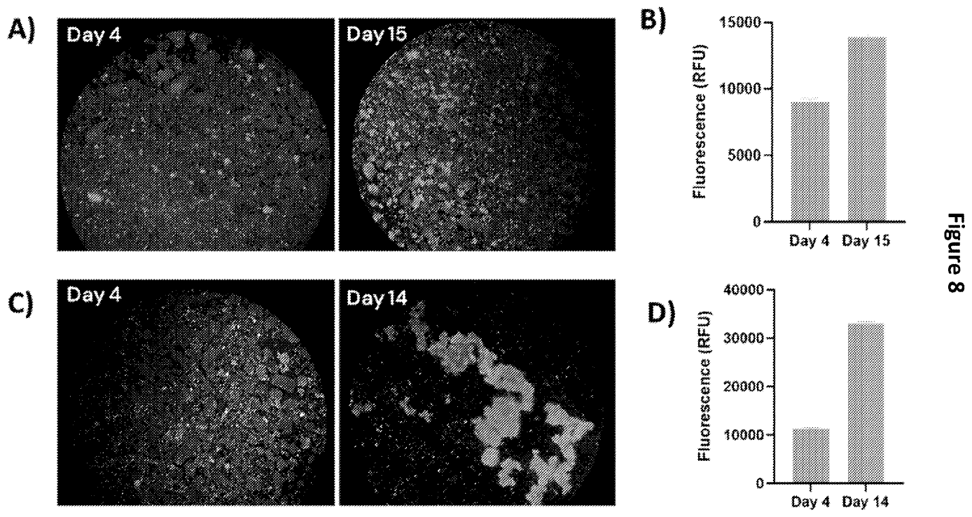

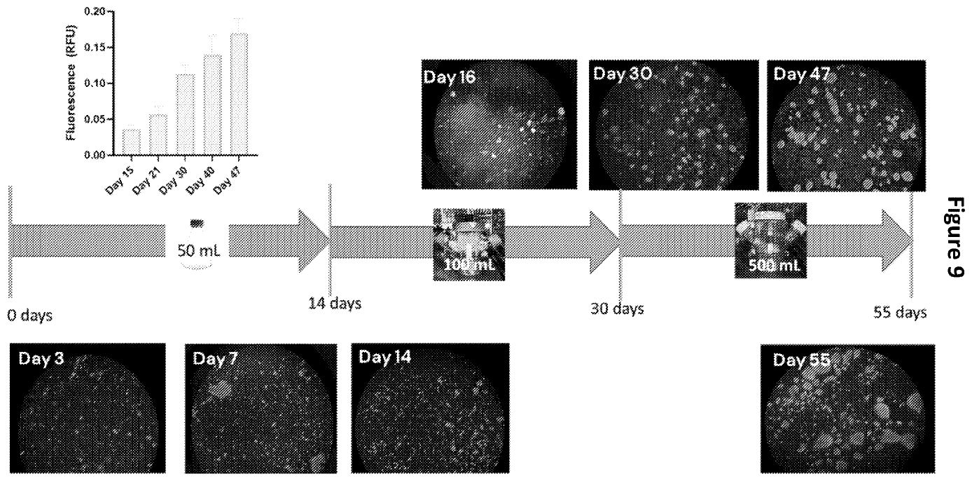

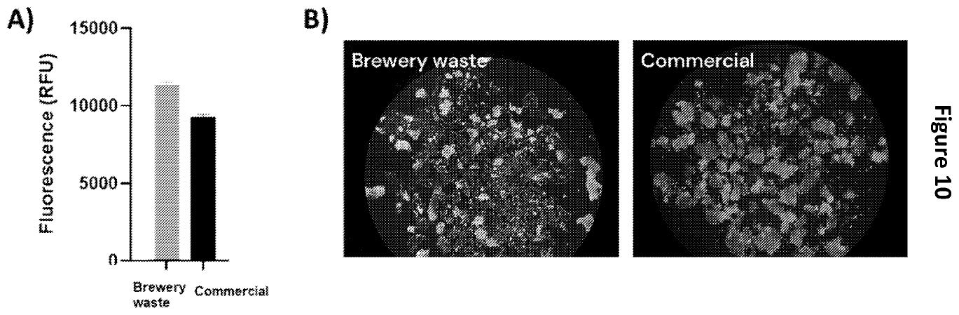

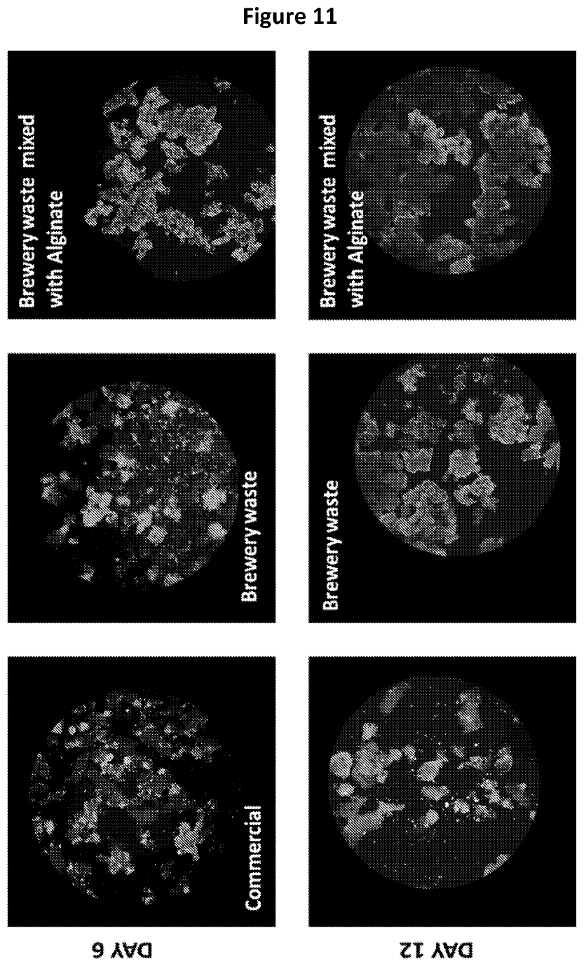

In some embodiments, the inactive yeast increases the metabolic activity of the animal cells or meat cells. Methods of determining metabolic activity of a cell are known on the art and exemplified in the Examples below, as shown for example in FIG. 2.

In some embodiments, the inactive yeast increases the metabolic activity of the animal cells or meat cells by about 1.5-fold or more. In some embodiments, the inactive yeast increases the metabolic activity of the animal cells or meat cells by about 2-fold or more. In some embodiments, the inactive yeast increases the metabolic activity of the animal cells or meat cells by about 3-fold or more. In some embodiments, the inactive yeast increases the metabolic activity of the animal cells or meat cells by about 4-fold or more. In some embodiments, the inactive yeast increases the metabolic activity of the animal cells or meat cells by about 5-fold or more. In some embodiments, the inactive yeast increases the metabolic activity of the animal cells or meat cells by about 6-fold or more. In some embodiments, the inactive yeast increases the metabolic activity of the animal cells or meat cells by about 7-fold or more. In some embodiments, the inactive yeast increases the metabolic activity of the animal cells or meat cells by about 8-fold or more. In some embodiments, the inactive yeast increases the metabolic activity of the animal cells or meat cells by about 9-fold or more. In some embodiments, the inactive yeast increases the metabolic activity of the animal cells or meat cells by about 10-fold or more. In some embodiments, the inactive yeast increases the metabolic activity of the animal cells or meat cells by about 20-fold or more.

In some embodiments, the inactivated yeast supports the metabolic activity of the animal cells in the presence of culture media comprising serum.

Surprisingly, the inventors have demonstrated that inactivated yeast supports the metabolic activity of the animal cells in the absence of serum in the culture medium. Accordingly, in some embodiments, the culture media does not comprise serum.

Methods of culturing animal cells for meat production, are well-known and described in the art.

For example, WO-A-2023/003471 describes a method for producing cultured fat cells for animal consumption. The method comprises the steps of:
providing a fibro-adipogenic progenitor (FAP) cell;
culturing said FAP cell in a culture medium for expanding FAP cells to thereby provide an expanded population of FAP cells; and
culturing said expanded population of FAP cells in a culture medium for differentiating FAP cells to thereby differentiate FAP cells into fat cells.

Such methods can be improved by including the culture ingredient described herein. For example, the present disclosure provides a method for producing cultured fat cells for animal consumption, comprising the steps of:
providing a fibro-adipogenic progenitor (FAP) cell;
culturing said FAP cell in a culture medium for expanding FAP cells to thereby provide an expanded population of FAP cells, wherein the culture medium comprises a culture ingredient that is composed of or produced by yeast, optionally wherein the yeast comprises inactivated yeast; and
culturing said expanded population of FAP cells in a culture medium for differentiating FAP cells to thereby differentiate FAP cells into fat cells, optionally wherein the culture medium comprises a culture ingredient that is composed of or produced by yeast, optionally wherein the yeast comprises inactivated yeast.

Animal cells that are adherent in culture typically require a physical support to survive. In some embodiments, the ingredient comprises a physical support for culturing animal cells or meat cells.

In some embodiments, the ingredient is formulated into a composite. In some embodiments, the ingredient is formulated into a composite that gels. In some embodiments, the ingredient is formulated into a composite that encapsulates. In some embodiments, the ingredient is formulated into a composite that dessicates. In some embodiments, the ingredient is formulated into a composite that preserves. In some embodiments, the ingredient is formulated into a composite that crystallises.

In some embodiments, the ingredient can be or is used as a base material. Typically, a base material refers to a material that animal cells attach to. The base material may coat a carrier, which can be macroscopic or microscopic. As will be understood by those in the art, macroscopic typically refers to objects that are visible to the naked eye. Accordingly, microscopic typically refers to objects that are not visible to the naked eye and require a microscope for observation. In some embodiments, the ingredient is used as a base material for coating a macrocarrier. In some embodiments, the ingredient is used as a base material for coating a microcarrier.

In some embodiments, the carrier comprises polystyrene. In some embodiments, the carrier comprises dextran. In some embodiments, the carrier comprises collagen. In some embodiments, the carrier comprises edible hydrogels.

The ingredient may be combined with a hydrogel for use as a physical support in methods described herein. Hydrogels can be formulated into desired geometries according to methods known in the art. It will be understood that the desired geometry may differ depending on the intended consumer meat product. In some embodiments, the hydrogel is formulated into a sphere. In some embodiments, the hydrogel is formulated into a disc. In some embodiments, the hydrogel is formulated into a toroid. In some embodiments, the hydrogel is formulated into a fibre. In some embodiments, the hydrogel is formulated into a tube.

Various hydrogels are described in the art. In some embodiments, the hydrogel comprises a polysaccharide hydrogel. In some embodiments, the hydrogel comprises alginate. In some embodiments, the hydrogel comprises pectin. In some embodiments, the hydrogel comprises gellan gum. In some embodiments, the hydrogel comprises kappa carrageenan. In some embodiments, the hydrogel comprises agarose. In some embodiments, the hydrogel comprises polyethylene glycol (PEG). In some embodiments, the PEG molecular weight is about 100 Da to about 20 kDa. In some embodiments, the PEG molecular weight is about 100 Da to about 10 kDa. In some embodiments, the PEG molecular weight is about 200 Da to about 5 kDa. In some embodiments, the PEG molecular weight is about 200 Da to about 1 kDa. In some embodiments, the PEG molecular weight is about 400 Da to about 1 kDa.

The ingredient can be combined with a hydrogel at various ratios. In some embodiments, the ingredient is combined with a hydrogel at a ratio of about 1:100. In some embodiments, the ingredient is combined with a hydrogel at a ratio of about 1:50. In some embodiments, the ingredient is combined with a hydrogel at a ratio of about 1:20. In some embodiments, the ingredient is combined with a hydrogel at a ratio of about 1:10. In some embodiments, the ingredient is combined with a hydrogel at a ratio of about 1:5. In some embodiments, the ingredient is combined with a hydrogel at a ratio of about 1:2. In some embodiments, the ingredient is combined with a hydrogel at a ratio of about 1:1.

Other methods described in the art provide hydrogels for the culturing animal cells, for example in WO-A-2021/158105, which describes a polysaccharide hydrogel. The polysaccharide hydrogel is a modified polysaccharide hydrogel comprising a low molecular weight alginate having a M w of 10 to 50 kDa and a M/G ratio of 0.8 to 1.5, wherein said alginate is conjugated with one or more cell-adhesion peptides, for use in cultured meat applications.

Accordingly, such hydrogels can be improved for the purpose of culturing animal cells, by coating the hydrogel with the culture ingredient described herein. For example, the present disclosure provides a modified polysaccharide hydrogel, comprising a low molecular weight alginate having a M w of 10 to 50 kDa and a M/G ratio of 0.8 to 1.5, wherein said alginate is conjugated with one or more cell-adhesion peptides, for use in cultured meat applications, wherein the alginate is coated with a culture ingredient that is composed of or produced by yeast, optionally wherein the yeast comprises inactivated yeast.

### Bioreactor

Methods described herein can be performed in a bioreactor. In some embodiments, a cell culture method as described herein is performed in a bioreactor. In some embodiments, the bioreactor comprises a stirred-tank. In some embodiments, the bioreactor comprises a rotating wall vessel. In some embodiments, the bioreactor comprises a fixed bed. In some embodiments, the bioreactor comprises a floating bed. In some embodiments, the bioreactor comprises a hollow fibre. In some embodiments, the bioreactor utilises rocking motion.

### Consumer Meat Product

Animal or meat cells cultured according to the methods described herein can be used in the formulation of a consumer meat product.

In some embodiments, the consumer meat product comprises at least one meat cell. In some embodiments, the at least one meat cell comprises a muscle cell. In some embodiments, the at least one meat cell comprises a fat cell. In some embodiments, the at least one meat cell comprises a connective tissue cell. In some embodiments, the at least one meat cell comprises a blood tissue cell.

In some embodiments, the consumer meat product comprises two or more meat cells. In some embodiments, the two or more meat cells comprise a muscle cell and a fat cell. In some embodiments, the two or more meat cells comprise a muscle cell and a connective tissue cell. In some embodiments, the two or more meat cells comprise a muscle cell and a blood cell. In some embodiments, the two or more meat cells comprise a fat cell and a connective tissue cell. In some embodiments, the two or more meat cells comprise a fat cell and a blood cell. In some embodiments, the two or more meat cells comprise a connective tissue cell and a blood cell.

In some embodiments, the consumer meat product comprises three or more meat cells. In some embodiments, the three or more meat cells comprise a muscle cell, a fat cell and a connective tissue cell. In some embodiments, the three or more meat cells comprise a muscle cell, a fat cell and a blood cell. In some embodiments, the three or more meat cells comprise a muscle cell, a connective tissue cell and a blood cell. In some embodiments, the three or more meat cells comprise a fat cell, a connective tissue cell and a blood cell.

In some embodiments, the consumer meat product comprises four or more meat cells. In some embodiments, the four or more meat cells comprise a muscle cell, a fat cell, a connective tissue cell and a blood cell.

In some embodiments, one, two, three, four, or more meat cells are co-cultured and extracted from the culture to form a consumer meat product. In other embodiments, meat cells are cultured separately, extracted from the culture, and combined in various ratios to form a consumer meat product. It will be understood that the ratio to be used will depend upon the consumer meat product being produced.

In some embodiments, the consumer meat product is selected from a burger, sausage, patty, chop, or steak. In some embodiments, the consumer meat product is selected from a burger, sausage, patty, chop, steak, paste, or fillet.

In some embodiments, the consumer meat product comprises the ingredient. In some embodiments, the ingredient is composed of or produced by yeast. In some embodiments, the ingredient is inactivated yeast. In some embodiments, the consumer meat product comprising the ingredient has an improved nutritional profile. In some embodiments, the consumer meat product comprising the ingredient has an improved nutritional profile compared to a comparable consumer meat product not comprising the ingredient. For example, a consumer meat product comprising the ingredient has increased vitamin B compared to a comparable consumer meat product not comprising the ingredient. For example, a consumer meat product comprising the ingredient has increased essential amino acids compared to a comparable consumer meat product not comprising the ingredient.

### Embodiments

The disclosure provides at least the following numbered embodiments are provided below, which are intended to define further the disclosed technologies but not intended to limit the scope of the invention, which is defined by the claims giving full account of equivalents.
1. A method for culturing animal cells using an ingredient that is composed of or produced by yeast, optionally wherein the ingredient comprises, consists essentially of or, or consists of, inactivated yeast.
2. A method for culturing animal cells according to claim 1 whereby the animal cells are grown in a bioreactor, wherein such bioreactor systems include, but are not limited to stirred-tank, rotating wall vessel, fixed bed, floating bed, hollow fibre, rocking motion.
3. A method for culturing animal cells according to claim 1 whereby the animal cells can be used to make edible products that can be used as a food substance for humans or other animals.
4. A method for culturing animal cells according to claim 1 whereby the animal cells can be or are from animal species that can be used to create edible material used as food or in food.
5. A method for culturing animal cells according to claim 3, whereby the animal species can include, but are not limited to cow, pig, sheep, goat, chicken, duck, turkey, deer, rabbit.
6. A method for culturing animal cells according to claim 1 whereby the ingredient is food-grade.
7. A method for culturing animal cells according to claim 1 whereby the ingredient is derived from the brewing process.
8. A method for culturing animal cells according to claim 1 whereby the ingredient is derived from the distillation process.
9. A method for culturing animal cells according to claim 1 whereby the ingredient can be supplemented with any other ingredient, nutrient, or additive to create usable formulations.
10. A method for culturing animal cells according to claim 1 whereby the ingredient can be used to supplement a cell culture process to provide physical and/or nutritional support to the cells for the purpose of creating substances that can be ingested.
11. A method for culturing animal cells according to claim 1 whereby the ingredient can be or is used in formulations with cells from the culture process to provide physical and/or nutritional support to the cells for the purpose of creating substances that can be ingested.
12. A method for culturing animal cells according to claim 1 whereby the ingredient can be or is formulated into a composite with an agent that gels, encapsulates, dessicates, preserves or crystalises.
13. A method for culturing animal cells according to claim 1 whereby the ingredient can be or is used as a base material for coating macro-and microcarriers for cell culture in suspension, where those macro-and microcarriers are made of materials including, but not limited to, polystyrene, dextran, collagen, and other edible hydrogel.
14. A method for culturing animal cells according to claim 1 whereby the ingredient can be or is used as a base material for coating hydrogels at macro or microscale formulated into geometries including, but not limited to disks, spheres, toroids, fibres, tubes.
15. A method for culturing animal cells according to claim 1 whereby:
   the ingredient can be or is used as a base material for the production of microcarriers; or
   a microcarrier comprises, consists of or consists essentially of the ingredient.
16. A method for culturing animal cells according to claim 1 whereby the ingredient can be or is used as a supplement to hydrogels including alginate, pectin, gellan gum, kappa carrageenan, agarose, peg, optionally wherein the supplemented hydrogel is used as a microcarriers or cells are encapsulated and grown inside them in 3D.
17. A method for culturing animal cells according to claim 16 whereby the ingredient can be or is used in combination with other nutritional components, to supplement culture media for physical and nutritional support.
18. A product obtainable or obtained by the method of any of claims 1 to 17.

19. A product according to claim 18, which is a consumer meat product, optionally selected from a burger, a sausage, a patty, a chop or a steak.
20. A product according to claim 18 or claim 19, comprising cultured meat cells and inactivated yeast flakes, yeast particles or yeast powder.
21. Use of inactivated yeast as a matrix, support, carrier or microcarrier for growing meat cells in in vitro culture, optionally wherein the meat cells comprise, consist essentially of or consist of muscle cells and/or fat cells.

### Examples

Although methods and materials similar or equivalent to those described herein can be used, suitable methods and materials are described below. The following examples are included for illustrative purposes only and are not intended to be limiting.

### Example 1

### Initial Preparation and Details

1. The nutritional yeast (NY) was obtained in the form of flakes from four different brands: Engevita, Purima, Special Ingredients, and Trzy Ziarna;
2. NY was manually ground into smaller particles using a mortar and pestle;
3. To prepare NY suspension, ground NY can either be autoclaved and then added to sterile culture medium separately, or be autoclaved together with the medium;
4. For the NY suspension, NY was added to culture media at 0.5% (g/ml). The suspension was prepared fresh prior to each experiment.

### Methodology for Cell Culture With Ny: Static Conditions

1. 750 μL of NY suspension was added to each well of a 24-well ultra-low attachment plate;
2. To each well, a cell suspension adjusted to a volume of 250 μL was added, so there was a total volume of 1 mL in each well;
3. The tested cell densities were 7500, 37500, and 75000 cells per mL (or 1.5, 7.5, and 15 million cells per g of NY)

### Methodology for Cell Culture With NY: Agitated Conditions

1. Cell culture with NY was tested in 50 mL shaker flasks, and 100 mL and 500 mL spinner flasks;
2. Sterile NY suspension was added to the flasks;
3. Cells were seeded to the flasks at different cell densities: 3×104, 1×105, and 1.94×105 cells/mL.

### Methodology for Encapsulation of Cells in Gels With Ny

1. Alginate solutions were prepared by adding 0.25%, 0.5%, 1%, and 2% alginate in ultrapure water;
2. A concentration of 0.5% (g/mL) of NY was added to each solution;
3. Solutions with added NY were autoclaved;
4. For cell encapsulation in the hydrogels, cells were counted and added to the solutions at a cell density of 106 cells per mL;
5. By using a pipette, drops with a volume of 10 μL of the solutions were pipetted into wells filled with 2% calcium lactate (CaLa) and incubated for 15 minutes;
6. CaLa was removed from the wells;
7. 1 mL of fresh culture medium was added to each well.

### Cell Types Tested

MyoSC, MSC and C2C12

### Example 2

### Initial Preparation Methods

1. The ingredient was either obtained in the form of flakes or powder from different commercial brands (Engevita, Purima, Special Ingredients, and Trzy Ziarna.) or it was sourced from waste products from breweries, cheese factories, whisky distillers and cider factories.
2. If sourced as powder or flakes from commercial sources, it was first manually ground into smaller particles using a mortar and pestle or any other method of grinding
3. If sourced from waste products from places such as but not limited to breweries, cheese factories, whisky distilleries and cider factories, the ingredient is sourced in a liquid form that is then processed into a powder/flake form.
4. First, multiple washes with water, and/or food grade solvents such as ethanol, isopropanol, methanol or any other permitted food extraction solvents or combination thereof would be carried out. This step could be carried out in static or agitated conditions, over a period ranging from 1 hour to 24 hours. The ratio of wet ingredient-to-solvent varies from 1:1 to 1:20.
5. The washed wet ingredient is then separated by using centrifugation, filtration, vacuum filtration or any other separation method or combination thereof.
6. The separated wet ingredient is then dried using either methods such as oven/vacuum oven drying, spray drying, freeze drying, hot air drying or any other method of drying at temperatures ranging from −80 °C to 150 °C
7. Modifications of the ingredient to improve cell adhesion and growth could also be achieved by mixing the ingredient with other components or materials. Examples of other components include proteins such as gelatine, collagen, laminins, fibronectin, Matrigel or any other proteins and protein coatings. The ingredient could be mixed as wet or dry in various proportions ranging from 1:1 to 1:20 with the proteins.
8. Other examples might include mixing the ingredient as wet or dry with hydrogels such as alginate, pectin, methylcellulose, gellan gum, carrageenans or any other hydrogels including food grade ones. The ingredient could be mixed with these hydrogels in proportions ranging from 1:1 to 1:20 with or without an additional step of crosslinking, The crosslinking of the hydrogel could be achieved using divalent ion sources such as calcium chloride, magnesium chloride, calcium lactate, magnesium lactate or any other divalent ion source. The hydrogels to be mixed could be in concentrations ranging from 0.2% wt to 20% wt.

### Methodology for Static and Suspension Cell Culture Using the Ingredient-based Microcarriers

1. For cell culture, the ingredient in solid form (flakes or powder) is first sterilised by using appropriate sterilisation methods including autoclaving, UV sterilisation, ethanol, or any other method of sterilisation.
2. The ingredient is weighted to achieve concentrations from 0.1% (g/ml) to 50% (g/ml) when resuspended in either serum-based or serum-free media formulations.
3. The ingredient is conditioned in the selected media formulations (either serum-based or serum-free) at the culture temperature ranging from 15 °C to 40 °C for a period ranging from 10 minutes to 12 hours prior to cell inoculation.
4. Cells are inoculated at densities ranging from 1,000 cells/cm² to 30,000 cells/cm² or 0.1×10⁶ cells/g of ingredient to 100×10⁶ cells/g of ingredient.
5. Multiple cell types can be cultured on the ingredient-based microcarrier including but not limited to myosatellite cells, adipose-derived stem cells, mesenchymal stem cells, fibro-adipogenic stem cells or other stem cell types from multiple species including bovine, porcine, ovine, chicken, turkey, water buffalo, mouse and any fish species including trout, salmon, tuna or any other fish species.
6. The ingredient-based microcarriers could be used to culture the cells mentioned at previous point in either non-treated well plates, non-treated flasks or bioreactors/fermenters at different scales.
7. The culture step in bioreactors could include regular media changes, complete or partial, with or without further additions of ingredient-based microcarriers.

It is understood that the Examples and embodiments described herein are for illustrative purposes only and that various modifications or changes in light thereof will be suggested to persons skilled in the art and are to be included within the spirit and purview of this application and scope of the appended claims.

All publications, sequence accession numbers, patents, and patent applications cited herein are hereby incorporated by reference in their entirety for all purposes.

1. A method for culturing animal cells in a culture medium comprising a culture ingredient that is composed of or produced by yeast.

2. The method according to claim 1, wherein the ingredient comprises, consists essentially of or, or consists of, inactivated yeast.

3. The method for culturing animal cells according to claim 1 wherein the animal cells are cultured in a bioreactor, wherein such bioreactor systems include, but are not limited to a stirred-tank, rotating wall vessel, fixed bed, floating bed, hollow fibre, or rocking motion bioreactor.

4. The method for culturing animal cells according to claim 1, wherein the animal cells are edible meat cells and can be used as a food substance for humans and/or non-human animals.

5. The method for culturing animal cells according to claim 1, wherein the animal cells are from species used to create edible material used as food or in food.

6. The method for culturing animal cells according to claim 1, wherein the animal species is cow, pig, sheep, goat, chicken, duck, turkey, deer, rabbit, trout, salmon, or tuna.

7. A method for culturing animal cells according to claim 1, wherein the ingredient is food-grade.

8. The method for culturing animal cells according to claim 1, wherein the ingredient is derived from a fermentation process, a brewing process or a distillation process, optionally wherein the ingredient is a waste-product from a fermentation process, a brewing process or a distillation process.

9. The method for culturing animal cells according to claim 1, wherein the ingredient is supplemented with at least one other ingredient, nutrient, or additive.

10. The method for culturing animal cells according to claim 1, wherein the ingredient supplements a cell culture process to provide physical and/or nutritional support to the animal cells for the purpose of creating substances that can be ingested.

11. The method for culturing animal cells according to claim 1, wherein the ingredient is used in formulations with cells from the culture process to provide physical and/or nutritional support to the cells for the purpose of creating substances that can be ingested.

12. The method for culturing animal cells according to claim 1, wherein the ingredient can be or is formulated into a composite with an agent that gels, encapsulates, dessicates, preserves or crystalises.

13. The method for culturing animal cells according to claim 1, wherein the ingredient can be or is used as a base material for coating macro- and microcarriers for cell culture in suspension, where those macro-and microcarriers are made of materials including, but not limited to, polystyrene, dextran, collagen, and other edible hydrogel.

14. The method for culturing animal cells according to claim 1, wherein the ingredient can be or is used as a base material for coating hydrogels at macro or microscale formulated into geometries including, but not limited to disks, spheres, toroids, fibres, tubes.

15. The method for culturing animal cells according to claim 1, wherein:
the ingredient can be or is used as a base material for the production of microcarriers; or
a microcarrier comprises, consists of or consists essentially of the ingredient.

16. The method for culturing animal cells according to claim 1, wherein the ingredient can be or is used as a supplement to hydrogels including alginate, pectin, gellan gum, kappa carrageenan, agarose, peg, optionally wherein the supplemented hydrogel is used as a microcarriers or cells are encapsulated and grown inside them in 3D.

17. The method for culturing animal cells according to claim 1, wherein the ingredient can be or is used in combination with other nutritional components, to supplement culture media for physical and nutritional support.

18. The product obtainable or obtained by the method of claim 1.

19. The product according to claim 18, which is a consumer meat product, optionally selected from a burger, a sausage, a patty, a chop or a steak.

20. The product according to claim 18, comprising cultured meat cells and inactivated yeast flakes, yeast particles or yeast powder.

21. A method of for growing meat cells in in vitro culture, comprising using inactivated yeast as a matrix, support, carrier or microcarrier, optionally wherein the meat cells comprise, consist essentially of or consist of muscle cells and/or fat cells.
